SEQUENCE LISTING (7 sequences)

SEQ ID NO: 1 - DNA, 15 bp, Artificial
  Beta Actin Foward Primer
tcaccgagcgcggct

SEQ ID NO: 2 - DNA, 21 bp, Artificial
  Beta Actin Reverse Primer
taatgtcacgcacgatttccc

SEQ ID NO: 3 - DNA, 22 bp, Artificial
  Beta Actin Probe
cagcttcaccaccacggccgag

SEQ ID NO: 4 - DNA, 21 bp, Artificial
  GC alpha 1 siRNA
ccuuguacauauaucagautt

SEQ ID NO: 5 - DNA, 21 bp, Artificial
  GC alpha 1 siRNA
ggcacccuuaaagauuuuutt

SEQ ID NO: 6 - DNA, 21 bp, Artificial
  GC alpha 2 siRNA
cgauacagcagacucucaatt

SEQ ID NO: 7 - DNA, 21 bp, Artificial
  GC alpha 2 siRNA
gcuaugcucugauguuucatt

(19) United States
(12) Patent Application Publication
(10) Pub. No.: US 2008/0176851 A1
(43) Pub. Date: Jul. 24, 2008

(54) Method for inhibiting lymphangiogenesis and inflammation

(75) Inventors: Kentaro Kajiya, Yokohama-shi (JP); Michael Detmar, Boppelsen (CH)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignees: Shiseido Company, Ltd., Tokyo (JP); ETH ZURICH, Zurich (CH)

(21) Appl. No.: 11/953,626

(22) Filed: Dec. 10, 2007

Related U.S. Application Data
(60) Provisional application No. 60/869,630, filed on Dec. 12, 2006.

(51) Int. Cl. A61K 31/5377; A61P 37/06

(52) U.S. Cl. 514/236.8

(57) ABSTRACT

A method for inhibiting lymphangiogenesis and/or inflammation by blocking sGCα1β1 signaling is provided. In a preferable embodiment, the blockage of sGCα1β1 signaling is accomplished by means of applying sGC inhibitor such as NS-2028 on skin.

### FIELD OF THE INVENTION

The present invention relates to a method for inhibiting lymphangiogenesis and inflammation.

### BACKGROUND ART

The lymphatic vascular system plays an important role in the maintenance of tissue fluid homeostasis, in the afferent phase of the immune response, and in the metastatic spread of cancers(1, 2). There is increasing evidence that lymphatic vessels also actively participate in acute and chronic inflammation. The chronic inflammatory skin disease psoriasis is characterized by pronounced cutaneous lymphatic hyperplasia, and chronic skin inflammation in mice is also associated with lymphatic endothelial cell (LEC) proliferation and lymphatic hyperplasia(3). Furthermore, kidney transplant rejection is frequently accompanied by lymphangiogenesis(4), and LEC-derived chemokines such as CCL21 might actively promote the inflammatory process(5). Lymphangiogenesis has also been observed in experimental models of chronic airway inflammation(6). Recently, we found that acute skin inflammation and edema formation induced by ultraviolet B (UVB) irradiation are associated with hyperpermeable, leaky lymphatic vessels that are functionally impaired(7). UVB irradiation of the skin also results in enhanced expression of vascular endothelial growth factor (VEGF)-A, and systemic blockade of VEGF-A led to diminished UVB-induced lymphatic vessel abnormalities and skin inflammation in mice, indicating that VEGF-A-mediated impairment of lymphatic vessel function promotes edema formation and inflammation. However, the mechanisms and functional consequences of lymphangiogenesis under inflammatory conditions are largely unknown.

Nitric oxide (NO) is a diatomic free radical molecule that is synthesized by a family of enzymes knows as nitric oxide synthases (NOS). There are three isoforms of NOS, including the calcium-dependent endothelial NOS (eNOS) and neuronal NOS (nNOS), and a calcium-independent inducible NOS (iNOS) (8). NO plays an important role as a regulatory mediator in a number of signaling processes. With regard to the vascular system, NO effects include vasodilation and enhanced vascular permeability(9). Soluble guanylate cyclase (sGC) is the only known physiological receptor for NO. Upon binding to NO, the activity of sGC is increased up to 400-fold, thereby promoting the conversion of GTP to cGMP and pyrophosphate. Synthesized cGMP regulates various effector proteins, including protein kinases, phosphodiesterases and ion channels(10). The sGC is a heme-containing heterodimeric protein consisting of 73- to 82-kDa alpha and 70-kDa beta-subunits(11). Two isotypes of human sGC have been identified(12). The originally identified human α3 and β3 subunits have now been reassigned as α1 and β1(13), and sGCα1β1 is mainly expressed in human heart and lung. The sGC α2/β2 isotype has been shown to localize to synaptic membranes in the brain(14). The sGC plays important roles in smooth muscle contractility, platelet reactivity, as well as in NO-induced hypotension in septic shock(15).

Recent studies have shown that NO is produced and released by lymphatic endothelial cells, possibly regulating lymphatic permeability and flow(16), and that stimulation of INOS activity in tumors is correlated with expression of the lymphangiogenic growth factor VEGF-C(17). However, little is known about the direct contributions of NO or of distinct sGC isoforms toward the control of normal and pathologic lymphatic vessel function.

### DISCLOSURE OF THE INVENTION

Because our previous gene array-based transcriptional profiling studies of cultured human lymphatic endothelial cells (LEC) versus blood vascular endothelial cells (BVEC) revealed highly increased expression levels of sGC subunits α1 and β1 in LEC as compared to BVEC, we further investigated the importance of the NO/sGC system for LEC function in vitro and in vivo. Here, we show that NO-induced LEC proliferation and migration are sGC-dependent, and that NO-induced cGMP production in LEC is specifically dependent on sGCα1β1. Importantly, the specific sGC inhibitor NS-2028 completely prevented ultraviolet B (UVB) irradiation-induced lymphatic vessel enlargement and skin inflammation and edema formation. These findings identify a crucial role of the NO/sGCα1β1/cGMP pathway in mediating lymphatic function. The blockade of NO/cGMP signaling might therefore serve as novel therapeutic strategy for inhibiting lymphangiogenesis and inflammation.

Accordingly, a method for inhibiting lymphangiogenesis and/or inflammation by blocking sGCα1β1 signaling is provided. In a preferable embodiment, the blockage of sGCα1β1 signaling is accomplished by means of applying sGC inhibitor such as NS-2028 (4H-8-Bromo-1,2,4-oxadiazolo(3,4-d)benz(b)(1,4)oxazin-1-one) on skin.

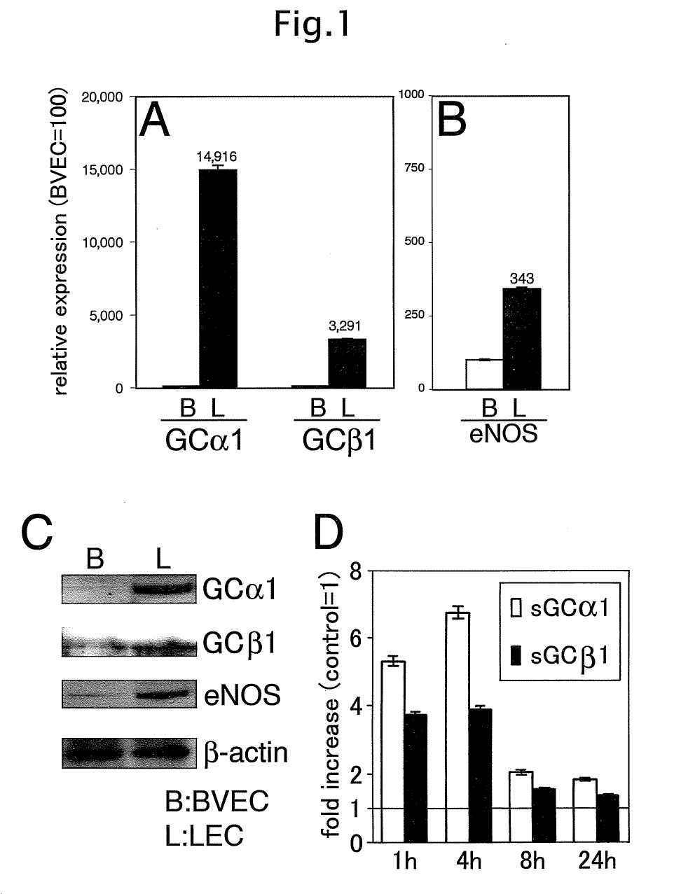

FIG. 1 shows enhanced expression of soluble guanylate cyclase α1 and β1 by lymphatic endothelial cells, as compared to blood vascular endothelial cells.

(A, B) Quantitative TaqMan real-time RT-PCR revealed that LEC (L; black bars) expressed more than 140-fold higher levels of sGCα1 mRNA (A) and more than 30-fold higher levels of sGCβ1 mRNA as compared to BEC (B; white bars). Expression of eNOS mRNA was increased by more than 3-fold in LEC (B). Results are expressed as mean value+/−SD (n=3) and are representative of the results obtained with three independent matched pairs of primary LEC and BVEC.

(C) Western blot analyses confirmed higher levels of sGCα1, sGCβ1 and eNOS protein expression in LEC as compared with BVEC. Equal levels of β-actin protein expression confirm equal loading.

(D) Quantitative real-time RT-PCR analysis of sGCα1 and sGCβ1 mRNA expression in LEC treated with 20 ng/ml VEGF-A for up to 24 h. VEGF-A treatment potently induced expression of both sGCα1 and sGCβ1 after 1 and 4 h. Data are expressed as mean values±SEM of three independent experiments.

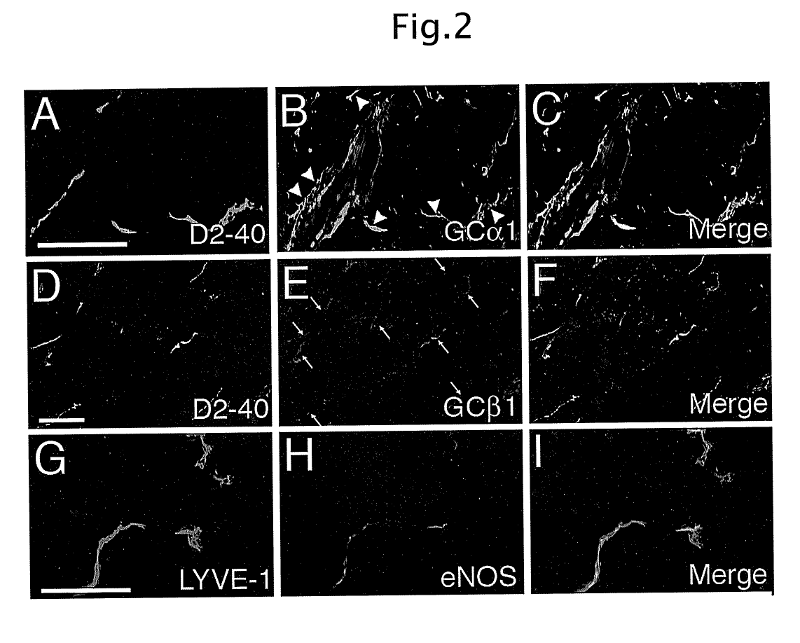

FIG. 2 shows expression of soluble guanylate cyclase α1 and β1 by cutaneous lymphatic vessels in situ.

(A-C) Double immunofluorescence analyses of human neonatal foreskin revealed that podoplanin-positive lymphatic vessels (D2-40; red) also expressed sGCα1 (green, arrowheads).

(D-F) sGCβ1 (red, arrows) was also expressed by D2-40-stained lymphatic vessels (green). (G-I) LYVE-1 positive lymphatic vessels (green) also expressed eNOS (red). Scale bars: 100 μm.

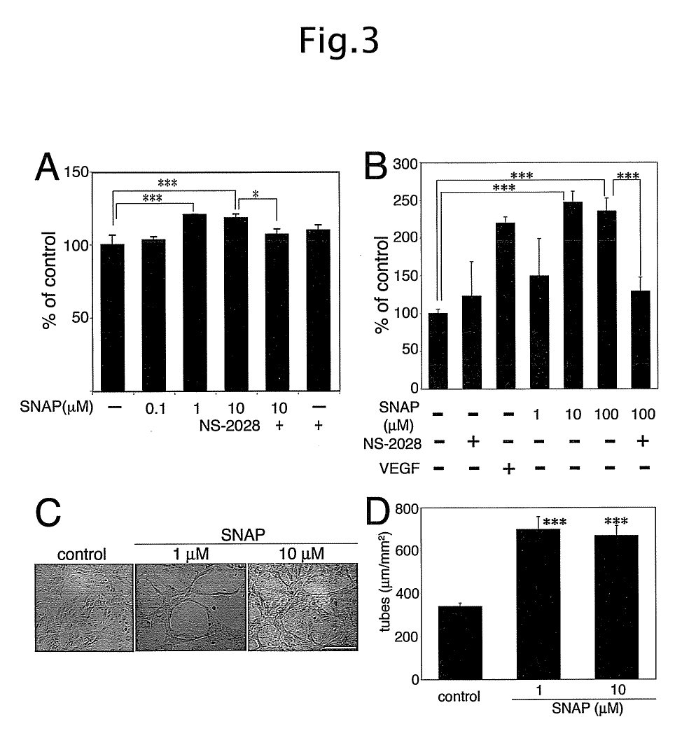

FIG. 3 shows that the NO-donor SNAP promotes LEC proliferation, migration and tube formation in a guanylate cylase dependent manner.

(A) The NO-donor SNAP induced proliferation of LEC with a minimal effective dose of 1 μM (p<0.001). The specific guanylate cyclase inhibitor NS-2028 significantly blocked the SNAP-induced proliferation (p<0.05).

(B) SNAP promoted migration of LEC with a minimal effective concentration of 1 μM (p<0.001). SNAP-induced LEC migration was blocked in the presence of NS-2028 (p<0.001).

(C, D) Treatment of LEC with 1 μM or 10 μM SNAP potently promoted the formation of tube-like structures in response to overlay with a type I collagen gel, as compared with untreated cultures (p<0.001). Data are expressed as mean values±SEM, n=5. Scale bar in panel C: 100 μm.

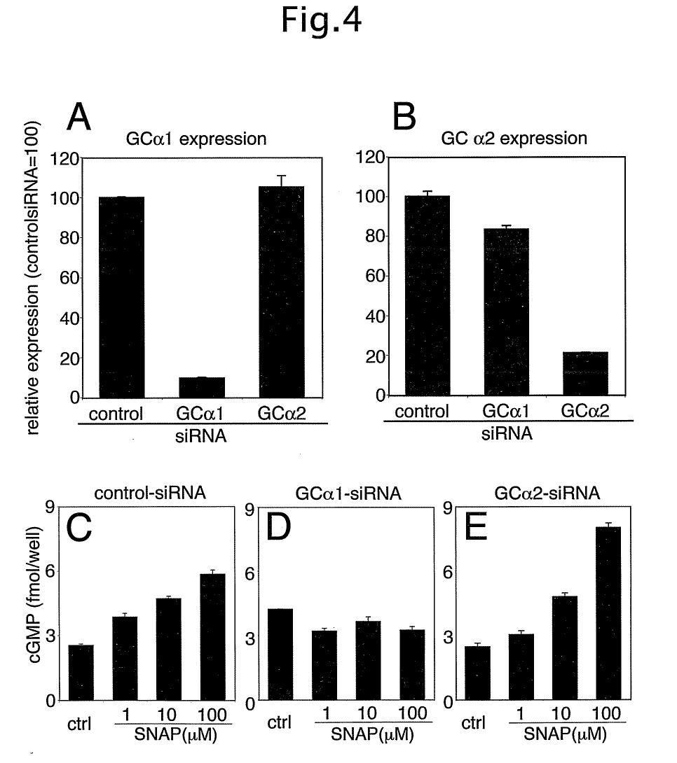

FIG. 4 shows that NO-induced cGMP production in LEC is dependent on sGCα1.

(A,B) siRNA-mediated knockdown of sGCα2 reduced expression of sGCα2 mRNA by more than 75% whereas GCα1 mRNA expression was unaffected. Conversely, sGCα1 but not sGCα2 mRNA expression was specifically knocked-down (by almost 90%) by sGCα1-targeted siRNA.

(C-E) The NO-donor SNAP dose-dependently induced cGMP production in control siRNA-transfected (C) and in sGCα2-siRNA transfected LEC (D). In contrast, no induction of cGMP was detected after siRNA-mediated knockdown of sGCα1 (E).

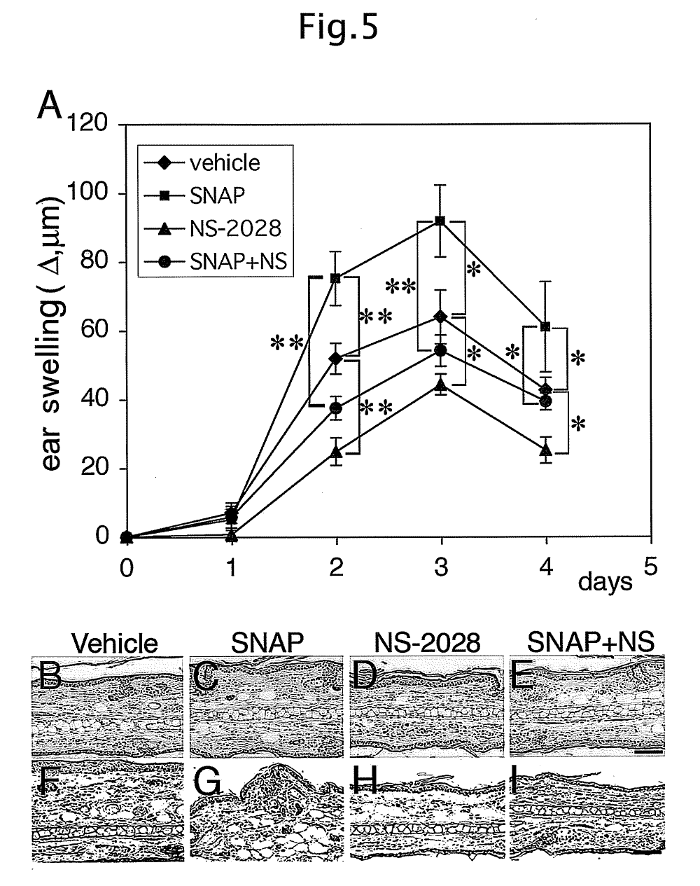

FIG. 5 shows that the guanylate cyclase inhibitor NS-2028 prevents UVB-induced skin inflammation.

(A) Pronounced ear swelling was clearly detectable in control-treated mice (υ) at 2 days after UVB irradiation with a maximal ear swelling at day 3. SNAP-treated mice (λ) showed significantly enhanced ear swelling (p<0.01 at day 2, p<0.05 at day 3 and 4), whereas ear swelling was significantly reduced in NS-2028 treated mice (σ). Combined treatment with SNAP and NS-2028 (λ) completely prevented the SNAP-induced augmentation of the UVB response. Ear swelling is expressed as the increase (Δ) in thickness (in μm) over pre-irradiation values. Data are expressed as mean±SEM (n=5). *p<0.05; **p<0.01.

(B-E) Histological analysis at day 4 after UVB irradiation revealed enhanced edema formation in the dermis of SNAP-treated mice (C), as compared with control-treated mice (B). In contrast, edema formation was decreased after NS-2028 treatment (D), and SNAP-induced edema formation was blocked in the presence of NS-2028 (E). B-E, hematoxylin-eosin stains. (F-I) Immunohistochemical stains for the macrophage/monocyte marker MOMA2 (brown) demonstrated inflammatory cell accumulation in the dermis of vehicle treated mice (F), whereas less MOMA-2 positive cells were found in NS-2028 treated mice (H). SNAP treatment resulted in strongly enhanced macrophage infiltration (G) that was blocked in the presence of NS-2028 (I). Scale bars, 100 μm.

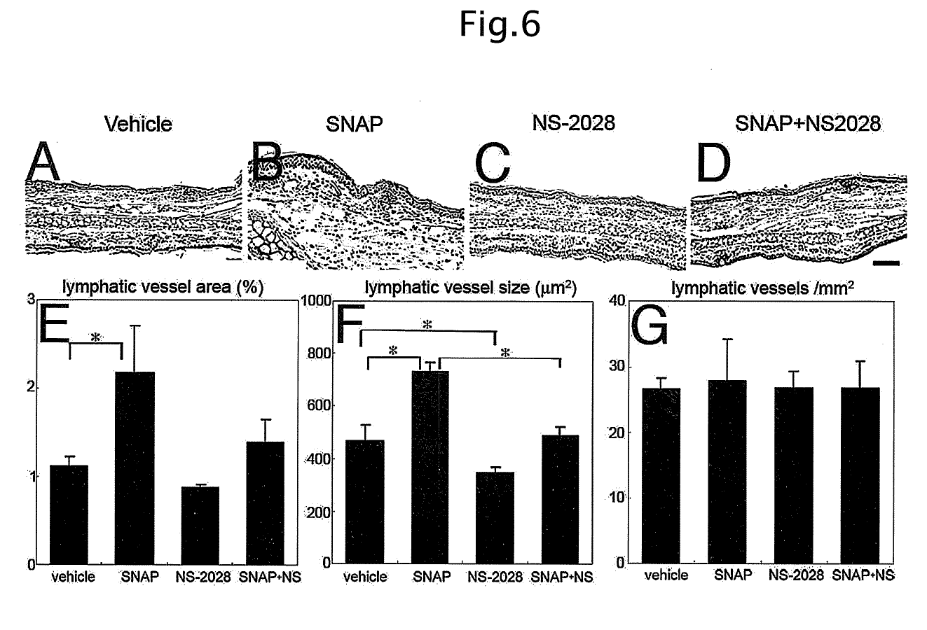

FIG. 6 shows that the guanylate cyclase inhibitor NS-2028 prevents inflammatory enlargement of lymphatic vessels.

(A-D) Immunohistochemistry for LYVE-1 revealed pronounced enlargement of lymphatic vessels in the dermis of SNAP-treated mice (B), as compared with vehicle-treated mice (A). NS-2028 treatment completely blocked the enlargement of lymphatic vessels (C). Treatment with NS-2028 also inhibited the SNAP-induced enlargement of lymphatic vessels (D). Scale bars, 100 μm. (E-G) Computer-assisted morphometric analyses of LYVE-1 stained sections revealed a significant increase of the average size of lymphatic vessels and of the area occupied by lymphatic vessels after SNAP treatment (E,F). Conversely, the average size of lymphatic vessels was significantly decreased after treatment with NS-2028 (F). The density of lymphatic vessels was comparable between the treatment groups (G). Data are expressed as mean±SEM (n=5). *p<0.05.

### BEST MODE FOR CARRYING OUT THE INVENTION

The present invention is based on the identifying the crucial role of the NO/sGCα1β1/cGMP pathway in mediating lymphatic function.

The present invention provides a method for inhibiting lymphangiogenesis and/or inflammation by blocking sGCα1β1 signaling. In a preferred embodiment, the blockage of sGCα1β1 signaling is accomplished by means of applying an sGC inhibitor such as NS-2028 (4H-8-Bromo-1,2,4-oxadiazolo(3,4-d)benz(b)(1,4)-oxazin-1-one) onto skin.

As the SGC inhibitor, a number of compounds are known in the art, and examples thereof include, but not limited to, NS-2028, ODQ (1H-[1,2,4]oxadiazolo[4,3-a]quinoxalin-1-one), LY83583 (6-anilino-5,8-quinolinedione) and the like. Preferable sGC inhibitor is NS-2028.

The above compounds themselves can be active ingredients for inhibiting lymphangiogenesis and/or inflammation by blocking sGCα1β1 signaling. The present invention not only ultimately leads to inhibition of lymphangiogenesis and/or inflammation, but can contribute to prophylaxis or treatment of dermatological diseases and cosmetic skin care.

The above compounds are used, as an active ingredient for a pharmaceutical or cosmetic composition for inhibiting lymphangiogenesis and/or inflammation by blocking sGCα1β1 signaling according to the invention, generally, as dry weight, in an amount of 0.00001 to 10% by weight, preferably 0.0001 to 5% by weight per weight of the total composition. At lower than 0.00001% by weight, the effects of the invention are hard to exert sufficiently, and even if it is compounded in an amount more than 10% by weight, so much enhancement of the effects is not attained and formulation becomes undesirably harder.

The pharmaceutical or cosmetic composition to be thus prescribed can be prepared by mixing or homogenizing the at least one of the above compounds into a suitable solvent, e.g., pure water, deionized water or buffered water, a lower alkanol such as methanol, ethanol or isopropyl alcohol or an aqueous solution thereof, glycerol or an aqueous solution thereof, a glycol such as propylene glycol or 1,3-butylene glycol or an aqueous solution thereof, or an oil such as hardened castor oil, vaseline or squalane, if necessary with use of a surfactant or the like. Into the composition can further appropriately be compounded, in such a range that the effects of the invention, that is, inhibition of lymphangiogenesis and/or inflammation by blocking sGCα1β1 signaling is/are not spoiled, other components usually used for external preparations such as cosmetics or pharmaceuticals, for example whitening agents, humectants, antioxidants, oily substances, ultraviolet absorbers, surfactants, thickeners, higher alcohols, powdery substances, colorants, aqueous substances, water, various skin nutrients, etc., according to necessity. Further, into the composition of the invention can appropriately be compounded sequestering agents such as disodium edetate, trisodium edetate, sodium citrate, sodium polyphosphate, sodium metaphosphate and gluconic acid, drugs such as caffeine, tannin, verapamil, tranexamic acid and its derivatives, grabridin, extract of fruit of Chinese quince with hot water, various crude drugs, tocopherol acetate, and glycylrrhetinic acid and its derivatives or salts, whiteners such as vitamin C, magnesium ascorbate phosphate, ascorbic acid glucoside, arbutin and kojic acid, saccharides such as glucose, fructose, mannose, sucrose and trehalose, vitamin A derivatives such as retinoic acid , retinol, retinol acetate and retinol palmitate, etc.

As to the above composition, its dosage form is not particularly limited, and can be any dosage forms such as solutions, solubilizing forms, emulsified forms, dispersed powders, water-oil two layer forms, water-oil-powder three layer forms, ointments, gels or aerosols. Its use form can also be optional, and can, for example be facial cosmetics such as skin lotion, liquid cream, cream and pack, foundation, and further makeup cosmetics, cosmetics for hair, aromatic cosmetics, bathing agents, etc., but is not limited thereto.

When the above composition is used on a living body, it can be endermically administered to local skin or the whole body skin of a subject. Its dose cannot be limited because the optimal amount varies depending on the age, sex and skin state of subjects, but, usually, it is sufficient that a composition prepared as mentioned above is administered onto the skin once or several times a day. If necessary, the dose or administration frequency can be determined referring to results obtained by evaluating a suitable specimen according to the evaluation method described later.

### EXAMPLES

### Materials and Methods

### Cells

Human dermal BVEC and LEC were isolated from neonatal human foreskins by immunomagnetic purification as previously described(18, 19). The lineage-specific differentiation was confirmed by real-time RT-PCR for the lymphatic vascular markers Prox1, LYVE-1 and podoplanin, and for the blood vascukar endothelial markers VEGF receptor-1 and VEGF-C, as well as by immunostains for CD31, Prox1 and podoplanin as described(18, 19). Cells were cultured in endothelial basal medium (Cambrex, Verviers, Belgium) supplemented with 20% fetal bovine serum (Gibco, Paisley, UK), antibiotics, 2 mM L-glutamine, 10 μg/ml hydrocortisone and 2.5×10⁻² mg/ml N-6,2-O-dibutyryl-adenosine 3′,5′-cyclic monophosphate (all from Fluka, Buchs, Switzerland) for up to eleven passages.

### Quantitative Real-Time RT-PCR

Total cellular RNA was isolated from confluent BVEC and LEC cultures at passage 5 using the Trizol reagent (Invitrogen, Carlsbad, Calif.). After treatment with RQ1 RNase-free-DNase (Promega, Madison, Wis.), the mRNA expression of vascular lineage-specific genes and of sGCα1, β1 and eNOS was investigated in triplicate by quantitative real-time TaqMan RT-PCR, using the ABI Prism 7000 Sequence Detection System (Applied Biosystems, Foster City, Calif.) and predesigned probes and primers (Applied Biosystems assay IDs Hs00181365_m1, Hs00168325_m1, Hs00167166_m1). Each reaction was multiplexed with β-actin primers (forward 5′-TCACCGAGCGCGGCT-3′, reverse 5′-TAATGTCACGCACGATTTCCC-3′) and probe (5′-JOE-CAGCTTCACCACCACGGCCGAG-TAMRA-3′) as an internal control. Triplicate LEC culture were also treated or not with 20 ng/ml of human recombinant VEGF-A165 (R&D Systems) for 1, 4, 8 or 24 h, followed by RNA extraction and real-time TaqMan RT-PCR analysis of sGCα1 and sGCβ1 expression.

### Immunoblotting

Western blot analyses of sGCα1, sGCβ1 and eNOS were performed as previously described. Briefly, confluent BVEC and LEC were homogenized in lysis buffer, and protein concentrations were determined using the BCA-Kit (Pierce Biotechnology, Rockford, Ill.). Equal amounts of lysates (100 μg protein) were immunoblotted with a rabbit polyclonal antibody against sGCα1 (Sigma, Saint Louis, Mo.), sGCβ1 (Cayman Chemical, Ann Arbor, Mich.) or eNOS (Santa Cruz Biotechnology, Santa Cruz, Calif.). Specific binding was detected by the enhanced chemiluminescence system (Amersham Biosciences, Piscataway, N.J.). Equal loading was confirmed with an antibody against β-actin (Sigma, St. Louis, Mo.).

### Immunostains

Immunofluorescence analysis was performed on 6-μm cryostat sections of human neonatal foreskins as described, using polyclonal antibodies against LYVE-1 (kindly provided by Dr. David Jackson, Oxford, UK), sGCα1, sGCβ1, eNOS (as described above), or a mouse monoclonal antibody against human podoplanin (20) (clone D2-40, Signet Laboratories, Dedham, Mass.), and corresponding secondary antibodies labeled with AlexaFluor488 or AlexaFluor594 (Molecular Probes, Eugene, Oreg.). Immunohistochemical analysis was performed on 5 μm AMEX-fixed sections as described(30), using antibodies against the macrophage monocyte marker MOMA-2 (BMA Biomedicals AG, Switzerland) or LYVE-1. Routine hematoxylin-eosin staining was also performed. Sections were examined by a Nikon E-600 microscope (Nikon, Melville, N.Y.) and images were captured with a SPOT digital camera (Diagnostic Instruments, Sterling Heights, Mich.). Computer-assisted morphometric analyses of representative LYVE-1-stained sections were performed as described(3).

### Proliferation, Migration and Tube Formation Assays

LEC (1.5×10³) were seeded into fibronectin-coated 96-well plates as described(19). For proliferation studies, quinduplicate wells were treated or not with different concentrations (0.1 to 10 μM) of the NO donor SNAP (S-Nitroso-N-acetyl-D,L-penicillamine) (Cayman Chemical, Ann Arbor, Mich.) in EBM containing 2% fetal bovine serum. In additional assays, LEC were incubated with 10 μM of SNAP, in the presence or absence of the specific guanylate cyclase inhibitor NS-2028 (100 nM, Cayman Chemical, Ann Arbor, Mich.). After 72 hours, cells were incubated with 5-methylumbelliferylheptanoate as described. The fluorescence intensity, proportional to the number of viable cells, was measured using a Spectra Max GEMINI EM (Bucher Biotech AG, Basel, Switzerland). Haptotatic cell migration was studied as described(19), using 24-well FluoroBlok inserts of 8 μm pore size (Falcon, Franklin Lakes, N.J.). Briefly, the bottom sides of the inserts were coated with 10 μg/ml fibronectin (BD Biosciences, Bedford, Mass.) for 1 hour, followed by incubation with 100 μg/ml bovine serum albumin. LEC (100 μl; 4×10⁵ cells/ml) in serum-free EBM containing 0.2% delipidized BSA were seeded into the upper chambers and were incubated for 3 hours at 37° C. in the presence of SNAP (0.1 to 10 μM). In additional migration studies, cells were pre-incubated with 100 nM of NS-2028 for 30 min, and were then seeded into the upper chambers in the presence of SNAP (10 μM) with or without 100 nM of NS-2028. After 3 hours incubation at 37° C., cells on the underside of the inserts were stained with Calcein-AM (Molecular Probes), and the fluorescence intensity, proportional to the number of viable cells, was measured using a Spectra Max GEMINI EM as described(19). Tube formation assays were performed as described. LEC were grown on fibronectin-coated 24-well plates until confluence. Then, 0.5 ml of a neutralized isotonic bovine dermal collagen type I solution (Vitrogen, Palo Alto, Calif.) with or without SNAP (1 or 10 μM) was added to the cells. After incubation at 37° C. for 6 hours, cells were fixed with 4% paraformaldehyde for 30 minutes at 4° C. Representative images were captured and the total length of tube-like structures per area was measured using the IP-LAB software as described(19). All studies were performed in triplicate. Statistical analyses were performed using the unpaired student's t-test.

siRNA Transfection and cGMP Immunoassay

siRNA transfection was performed using the Basic Nucleofactor Kit for primary mammalian endothelial cells (Amaxa Biosystems, CITY, Germany). Briefly, after trypsinization, LEC (5×10⁵) were resuspended in 100 μl of basic nucleofactor solution. Cells were transfected by electroporation (Nucleofactor II, Amaxa Biosystems), using 1.6 μg of siRNA containing two different double-stranded oligonucleotides for each sGCα2 or sGCα1. The following siRNAs were used: GCα1: CCUUGUACAUAUAUCAGAUtt and GGCACCCUUAAAGAUUUUUtt, GCα2: CGAUACAGCAGACUCUCAAtt and GCUAUGCUCUGAUGUUUCAtt. Control siRNA (Silencer negative control #1 siRNA, Ambion, Cambridgeshire, UK) comprising a 19-bp scrambled sequence with 3′dT overhangs was used as a control; the sequences has no significant sequence homology to any known gene sequence. At 72 hours after tranfection, cells were used for RNA purification or for cGMP assays. Efficient knockdown of mRNA expression was confirmed by TaqMan real-time RT-PCR for sGCα1 and sGCα2 (Applied Biosystems assay ID for sGCα2 was Hs00181365_ml). For cGMP enzyme immunoassays, 1×10⁴ siRNA-transfected LEC were seeded into individual wells of a 96-well plate and were incubated with 2% FBS-containing medium. After 24 hours, cells were incubated with several concentrations of SNAP (1-100 μM) for 15 minutes. The cellular cGMP concentrations were measured using an EIA immunoassay kit (Amersham Biosciences, Freiburg, Germany).

### UVB Irradiation Regimen

A total of 15 female albino HR-1 hairless mice (Hoshino Laboratory Animal Co., Ltd, Japan) at 8 weeks of age were exposed to a single dose of 200 mJ/cm² of UVB irradiation, using ten Toshiba FL-20 SE fluorescent lamps that deliver energy in the UVB (280-340 nm) wave length range with a maximum energy at a wavelength of 305 nm as described(31). Beginning one day before UVB irradiation, the right ears were daily treated topically with 1 mM of SNAP, 10 μM of NS-2028, or 1 mM of SNAP together with 100 μl of 10 μM of NS-2028 in a 50% ethanol solution (n=5 per group) until day 3 after UVB irradiation (5 times total). The left ears were treated with vehicle only. The thickness of the ears was measured every day until day 4 after the UVB irradiation. At 4 days after the UVB irradiation, mouse ears were fixed in cold acetone (AMeX procedure) (30)and were processed for histological analysis of paraffin sections. All animal studies were approved by the Shiseido Life Science Research Center Committee on Research Animal Care.

### Results

### Increased Expression of Soluble Guanylate Cyclase α1β1 by Cultured LEC as Compared to BVEC

To identify genes that are specifically expressed or upregulated by LEC, compared to BVEC, we isolated both LEC and BVEC from human neonatal foreskins of three independent donors, as previously described. The three LEC and of BVEC cell lines were then subjected to transcriptional profiling by microarray analysis, using Affymetrix HU133 plus 2.0 arrays(18, 19). These studies revealed that—among several other genes(19)—soluble guanylate cyclase (sGC) α1 was expressed at much higher levels by LEC than by BVEC, with an average fold increase in LEC over BVEC of 14.9±3.48. Expression of sGC β1 (2.09±1.5 fold) and of eNOS (13.0±1.8 fold) was also increased in LEC, whereas expression levels of iNOS, sGCα2 and sGCβ2 were below detection level. These findings were confirmed by quantitative TaqMan real-time RT-PCR, revealing a more than 140-fold upregulation of sGCα1, a more than 30-fold upregulation of sGCβ1 (FIG. 1A), and a 3-fold increase of eNOS expression in LEC as compared with BVEC (FIG. 1B). Western blot analyses demonstrated that also the protein expression of sGCα1, sGCβ1, and eNOS was strongly increased in LEC (FIG. 1C). Expression of sGCα1 and sGCβ mRNA was strongly increased by treatment of LEC with 20 ng/ml of VEGF-A, with a more than 6-fold upregulation of sGCα1expression after 4 h (FIG. 1D).

sGCα1, sGCβ1, and eNOS are expressed by lymphatic vessels in situ.

We next performed differential immunofluorescence analyses of lymphatic vessels in frozen sections of human neonatal foreskin samples. Lymphatic vessels were specifically detected by using the anti-human podoplanin antibody D2-40(20) or an antibody against the lymphatic-specific hyaluronan receptor LYVE-1(21). The majority of D2-40 positive lymphatic vessels also showed immunoreactivity for sGCα1 (FIG. 2A-C) and sGCβ1 (FIG. 2 D-F). As expected, both sGCα1 and sGCβ1 were also expressed by epidermal keratinocytes (data not shown). Lymphatic vessels also expressed eNOS (FIG. 2G-I).

Nitric oxide-induced LEC proliferation and migration are dependent on Guanylate cyclase.

To further characterize the effects of nitric oxide (NO) on LEC functions, LEC were treated with the NO donor SNAP. We found that SNAP dose-dependently induced LEC proliferation with a minimal effective concentration of 1 μM (p<0.001; FIG. 3A). SNAP-induced proliferation was significantly blocked in the presence of the selective guanylate cyclase inhibitor NS-2028 (p<0.05), whereas NS-2028 itself did not have any effect on untreated LEC (FIG. 3A). SNAP treatment also promoted haptotactic migration of LEC—as potently as the established (lymph)angiogenic factor VEGF-A—, with a minimal effective concentration of 10 μM (p<0.001; FIG. 3B). Addition of NS-2028 strongly inhibited the promigratory effect of SNAP (p<0.001). After overlay of confluent LEC cultures with a type I collagen gel, SNAP also potently induced in vitro cord formation by LEC with a minimal effective dose of 1 μM at 6 hours (p<0.001; FIG. 3 C,D).

NO-induced cGMP production in LEC is dependent on sGCα1.

Binding of NO to guanylate cyclase results in the catalysis of GTP to cGMP(22). To investigate which subtype of sGCα is responsible for mediating NO effects in LEC, we studied NO-mediated cGMP production after specific, siRNA-mediated knockdown of sGCα1 or sGCα2. Quantitative real-time RT-PCR confirmed a specific, more than 85% knockdown of sGCα1 but no significant change of sGCα2 expression at 48 h after transfection with sGCα1-specific siRNA, compared to control siRNA transfection, whereas sGCα2 knockdown specifically decreased the expression of sGCα2, but not of sGCα1, by more than 90% (FIG. 4A,B). Control siRNA-transfected and sGCα2 siRNA-tranfected LEC dose-dependently responded to SNAP treatment with increased cGMP production, at a minimal effective dose of 1 μM (FIG. 4C,D), whereas induction of cGMP production by SNAP was completely prevented in sGCα1-siRNA transfected LEC (FIG. 4E).

UVB-induced edema formation and skin inflammation are promoted by the NO donor SNAP and are inhibited by the guanylate cyclase inhibitor NS-2028.

We have previously shown that UVB irradiation of the skin results in enhanced expression of VEGF-A, associated with edema, inflammation and lymphatic vessel enlargement and leakiness(7). Moreover, systemic blockade of VEGF-A led to diminished UVB-induced lymphatic vessel abnormalities and skin inflammation in mice, indicating that VEGF-mediated impairment of lymphatic vessel function promotes UVB-induced inflammation. Because VEGF-A treatment of LEC potently induced the expression of sGCα1β1, we next investigated whether NO plays a role in lymphatic function in vivo, and whether inhibition of guanylate cyclase activity in vivo might reduce UVB-induced skin damage. To this end, HR-1 hairless mice were exposed to a single dose of 200 mJ/cm² of UVB irradiation. Beginning one day before irradiation, the right ears of the mice were treated daily with topical application of the NO donor SNAP, the guanylate cyclase inhibitor NS-2028 NAP in combination with NS-2028, or with vehicle only. Ear thickness was measured daily as a parameter for skin inflammation and edema formation. Skin inflammation and edema formation were clearly detectable in control-treated mice at 2 days after UVB irradiation with a maximal ear swelling at day 3 (FIG. 5A). SNAP-treated mice showed significantly enhanced ear swelling (p<0.01 at day 2, p<0.05 at day 3 and 4), whereas ear swelling was significantly reduced in NS-2028 treated mice (p<0.01 at day 2, p<0.05 day 3 and 4) (FIG. 5A). Combined treatment with SNAP and NS-2028 completely prevented the SNAP-induced augmentation of the UVB response (p<0.01 at day 2 and 3, p<0.05 at 4).

Histological analysis at day 4 after UVB irradiation revealed enhanced edema formation in the dermis of SNAP-treated mice (FIG. 5C), as compared with control-treated mice (FIG. 5B). In contrast, edema formation was decreased after NS-2028 treatment (FIG. 5D), and SNAP-induced edema formation was blocked in the presence of NS-2028 (FIG. 5E). Immunohistochemical stains for the macrophage/monocyte marker MOMA2 demonstrated inflammatory cell accumulation in the dermis of vehicle treated mice (FIG. 5F), whereas less MOMA-2 positive cells were found in NS-2028 treated mice (FIG. 5H). SNAP treatment resulted in strongly enhanced macrophage infiltration (FIG. 5G) that was blocked in the presence of NS-2028 (FIG. 5I).

We next investigated the effects of guanylate cyclase inhibition on the number and size of cutaneous lymphatic vessels by immunohistochemistry, using an antibody against the lymphatic specific hyaluronan-receptor LYVE-1. In agreement with our previous studies, we found enlargement of lymphatic vessels in UVB-irradiated mice that received control vehicle treatment (FIG. 6A). There was a pronounced enlargement of lymphatic vessels in the dermis of SNAP-treated mice (FIG. 6B), as compared with vehicle-treated control mice (FIG. 6A), whereas NS-2028 treatment completely blocked the enlargement of lymphatic vessels (FIG. 6C). Treatment with NS-2028 also inhibited the SNAP-induced enlargement of lymphatic vessels (FIG. 6D). Computer-assisted morphometric analyses of LYVE-1 stained sections confirmed these findings and revealed a significant increase of the average size of lymphatic vessels (730.7 μm²±34.9 m²; p<0.05) and of the area occupied by lymphatic vessels (2.18%±0.52%; p<0.05) after SNAP treatment (FIG. 6E,F). Conversely, the average size of lymphatic vessels was significantly decreased after treatment with NS-2028 (350.1 μm²±19.5 μm²; p<0.05; FIG. 6F). The density of lymphatic vessels was comparable between the treatment groups (FIG. 6G). Together, these findings reveal an important role of the NO/sGCα1β1/cGMP pathway in mediating lymphatic vessel function in inflammation.

### Discussion

In a search for mediators of lymphangiogenesis, we have used gene expression analysis, in vitro and in vivo studies to identify soluble guanylate cyclase α1β1 as an important mediator of lymphatic vessel function. We found that sGCα1is expressed much more strongly by cultured LEC than by BVEC, that VEGF-A potently induces LEC expression of sGCα1, that NO-induced LEC proliferation and migration are dependent on soluble guanylate cyclase activity, and that sGCα1 is the only receptor for mediating nitric oxide effects on LEC cGMP production. Furthermore, NO promotes lymphatic vessel dilation and edema in vivo, and UVB-induced lymphatic vessel dilation and skin inflammation are potently blocked by inhibiting soluble guanylate cyclase. Blockade of soluble guanylate cyclase might therefore serve as a new strategy to improve lymphatic drainage and to inhibit inflammation.

There has been a recent surge of interest in the lymphatic vascular system, mainly because of its emerging active role in lymphatic tumor metastasis(23). The quest to identify lymphatic-specific growth factors and differentiation markers has been hampered, however, by the lack of reliable markers to distinguish between the lymphatic and the blood vascular endothelial cell lineage. The recent identification of several lymphatic specific genes, including Prox1, LYVE-1 and podoplanin, has now cleared the path for molecular investigations of lymphatic lineage-specific differentiation and function, and also for the reliable isolation and comparison of LEC and BVEC. Thus, we have been able to perform comparative transcriptional profiling of these cells and to identify novel lymphatic lineage signature genes(18). Because our comparison of several primary cell lines revealed that sGCα1 is one of the most strongly LEC-specific genes, and because of the established importance of the nitric oxide pathway in blood vascular physiology and pathology(9), we further investigated key molecular players of the NO/cGMP pathway and their functional relevance in LEC.

We found that—in addition to sGCα1—also sGCβ1 and eNOS, but not sGCα2, sGCβ2 or iNOS were more strongly expressed by LEC than by BVEC, as revealed by quantitative real-time RT-PCR and western blotting. The sGC occurs in two isoforms, either sGCα1β1 or sGCα2β2(24). Our results indicate that sGCα1β1 is the only functional NO receptor in LEC, because specific knock-down of sGCα1 completely inhibited the cGMP production induced by the NO donor SNAP in LEC, whereas SNAP-induced cGMP production was not affected by knockdown of sGCα2. Moreover, LEC proliferation and migration were enhanced by incubation with the NO donor SNAP, and this NO induced effect could be completely abolished by treatment with the guanylate cyclase inhibitor NS-2028. These findings indicate that NO promotes LEC proliferation and migration via the sGCα1β1 and cGMP pathway—and not via cGMP-independent pathways as has been recently reported for the VEGF-E effect on human umbilical vein endothelial cell migration(25).

NO has been shown to exert potent vasoactive effects. In arteries, activation of eNOS results in enhanced diffusion of NO to the underlying vascular smooth muscle cells, where it stimulates sGC to produce more cGMP, leading to actin disassembly and vasodilation. In contrast, lymphatic capillaries are not ensheathed by pericytes or smooth muscle cells, but consist of a single layer of lymphatic endothelial cells (reviewed in (26)). In the presence of increased interstitial fluid pressure, usually caused by increased leakage from blood vessels, lymphatic capillaries are thought to be passively “pulled” open by anchoring filaments that directly connect the lymphatic endothelial cells with elastic fibers in the extracellular matrix—possibly facilitating fluid drainage via the lymphatic vascular system. However, our findings as well as previous studies suggest that lymphatic vessel dilation might also be actively induced by mediators that act directly on LEC(16). Because we found that sGCα1β1 is selectively expressed by cultured LEC, as compared to BVEC, and also by lymphatic capillaries in the skin, and because NO exerts direct effects on LEC migration and tube formation in vitro, it is tempting to speculate that activation of the NO pathway might also directly target LEC to induce lymphatic capillary dilation in vivo. In addition, NO may negatively affect the pumping activity of larger, collecting lymphatic vessels further contributing to lymphatic capillary dilation via lymphatic stasis(27). In future studies, it will be of interest to investigate the effects of NO on the cytoskeletal rearrangement of LEC and the interaction between anchoring filaments and the extracellular matrix.

Edema is a cardinal feature of inflammatory diseases and results when the amount of fluid leakage from inflamed blood vessels exceeds the capacity of lymphatic vessels for drainage(6). Whereas abundant research efforts have characterized the molecular control of blood vascular permeability, little is known about the control mechanisms regulating lymphatic vessel function in inflammation. Previously, we have shown that lymphatic vessels play an important role in UVB-induced edema and skin inflammation, and that UVB irradiation leads to enlarged and leaky lymphatic vessels(7, 28). Importantly, the UVB effect on lymphatic vessels was mediated by VEGF-A because systemic inhibition of VEGF-A prevented UVB-induced edema and lymphatic enlargement whereas overexpression promoted edema formation and lymphatic dysfunction VEGF-A(7). Our present study identifies the NO/sGCα1β1/cGMP pathway as an important mediator of VEGF-A's effects on lymphatic vessel function in inflammation. VEGF-A promotes this pathway by at least two distinct mechanisms since VEGF-A treatment of LEC—in addition to enhancing sGCα1β1 expression—also increased expression of iNOS and thereby NO production (data not shown). Blockade of sGC by NS-2028 completely prevented UVB-induced lymphatic enlargement and edema formation, indicating that VEGF-A's effects are dependent on cGMP production, and suggesting that inhibition of this pathway in lymphatic endothelium might serve as a novel strategy for inhibiting inflammation. Because high eNOS activity has been linked to tumor progression(29), it will be of interest to investigate whether the NO/sGCα1β1/cGMP axis is also involved in mediating tumor-associated lymphangiogenesis and lymphatic cancer metastasis.

### REFERENCES

1. Alitalo, K., Tammela, T. & Petrova, T. V. (2005) Nature 438, 946-53.
2. Oliver, G. & Detmar, M. (2002) Genes Dev 16, 773-83.
3. Kunstfeld, R., Hirakawa, S., Hong, Y. K., Schacht, V., Lange-Asschenfeldt, B., Velasco, P., Lin, C., Fiebiger, E., Wei, X., Wu, Y., Hicklin, D., Bohlen, P. & Detmar, M. (2004) Blood 104, 1048-57.
4. Kerjaschki, D., Huttary, N., Raab, I., Regele, H., Bojarski-Nagy, K., Bartel, G., Krober, S. M., Greinix, H., Rosenmaier, A., Karlhofer, F., Wick, N. & Mazal, P. R. (2006) Nat Med 12, 230-4.
5. MartIn-Fontecha, A., Sebastiani, S., Hopken, U. E., Uguccioni, M., Lipp, M., Lanzavecchia, A. & Sallusto, F. (2003) J Exp Med 198, 615-21.
6. Baluk, P., Tammela, T., Ator, E., Lyubynska, N., Achen, M. G., Hicklin, D. J., Jeltsch, M., Petrova, T. V., Pytowski, B., Stacker, S. A., Yla-Herttuala, S., Jackson, D. G., Alitalo, K. & McDonald, D. M. (2005) J Clin Invest 115, 247-57.
7. Kajiya, K., Hirakawa, S., Detmar, M. Am J Pathol in press.
8. Stuehr, D. J. (1999) Biochim Biophys Acta 1411, 217-30.
9. Kim-Shapiro, D. B., Schechter, A. N. & Gladwin, M. T. (2006) Arterioscler Thromb Vasc Biol 26, 697-705.
10. Lee, Y. C., Martin, E. & Murad, F. (2000) Proc Natl Acad Sci USA 97, 10763-8.
11. Boon, E. M., Huang, S. H. & Marletta, M. A. (2005) Nat Chem Biol 1, 53-9.
12. Kosarikov, D. N., Young, P., Uversky, V. N. & Gerber, N. C. (2001) Arch Biochem Biophys 388, 185-97.
13. Zabel, U., Weeger, M., La, M. & Schmidt, H. H. (1998) Biochem J 335 (Pt 1), 51-7.
14. Yuen, P. S., Potter, L. R. & Garbers, D. L. (1990) Biochemistry 29, 10872-8.
15. Huston, W. M., Andrew, C. R., Servid, A. E., McKay, A. L., Leech, A. P., Butler, C. S. & Moir, J. W. (2006) Biochemistry 45, 4388-95.
16. Ohhashi, T., Mizuno, R., Ikomi, F. & Kawai, Y. (2005) Pharmacol Ther 105, 165-88.
17. Franchi, A., Massi, D., Santucci, M., Masini, E., Degl'Innocenti, D. R., Magnelli, L., Fanti, E., Naldini, A., Ardinghi, C., Carraro, F. & Gallo, O. (2006) J Pathol 208, 439-45.
18. Hirakawa, S., Hong, Y. K., Harvey, N., Schacht, V., Matsuda, K., Libermann, T. & Detmar, M. (2003) Am J Pathol 162, 575-86.
19. Kajiya, K., Hirakawa, S., Ma, B., Drinnenberg, I. & Detmar, M. (2005) EMBO J 24, 2885-95.
20. Schacht, V., Dadras, S. S., Johnson, L. A., Jackson, D. G., Hong, Y. K. & Detmar, M. (2005) Am J Pathol 166, 913-21.
21. Banerji, S., Ni, J., Wang, S. X., Clasper, S., Su, J., Tammi, R., Jones, M. & Jackson, D. G. (1999) J Cell Biol 144, 789-801.
22. Denninger, J. W. & Marletta, M. A. (1999) Biochim Biophys Acta 1411, 334-50.
23. Tobler, N. E. & Detmar, M. (2006) J Leukoc Biol.
24. Russwurm, M., Wittau, N. & Koesling, D. (2001) J Biol Chem 276, 44647-52.
25. Cudmore, M., Ahmad, S., Al-Ani, B., Hewett, P., Ahmed, S. & Ahmed, A. (2006) Biochem Biophys Res Commun 345, 1275-82.
26. Cueni, L. Detmar, M. J Invest Dermatol. in press
27. Shirasawa, Y., Ikomi, F. & Ohhashi, T. (2000) Am J Physiol Gastrointest Liver Physiol 278, G551-6.
28. Kajiya, K. & Detmar, M. (2006) J Invest Dermatol 126, 919-21.
29. Jadeski, L. C., Hum, K. O., Chakraborty, C. & Lala, P. K. (2000) Int J Cancer 86, 30-9.
30. Sato, Y., Mukai, K., Watanabe, S., Goto, M. & Shimosato, Y. (1986) Am J Pathol 125, 431-5.
31. Yano, K., Kajiya, K., Ishiwata, M., Hong, Y. K., Miyakawa, T. & Detmar, M. (2004) J Invest Dermatol 122, 201-8.

What is claimed is:

1. A method for inhibiting lymphangiogenesis and/or inflammation by blocking sGCα1β1 signaling.

2. The method according to claim 1 wherein the blockage of sGCα1β1 signaling is accomplished by means of applying an sGC inhibitor onto skin.

3. The method according to claim 2 wherein said sGC inhibitor is NS-2028.
